(19) United States
(12) Patent Application Publication
(10) Pub. No.: US 2022/0032297 A1
(43) Pub. Date: Feb. 3, 2022

(54) MICROFLUIDIC METHOD FOR ANALYZING METALS

(71) Applicant: KLEARIA, VALBONNE (FR)

(72) Inventors: Floriant DOUNGMENE, NICE (FR); Lisa GAUTRIN, GRASSE (FR); Aurélie MAGNIER, ORLÉANS (FR); Clément NANTEUIL, CAGNES SUR MER (FR)

(73) Assignee: KLEARIA, VALBONNE (FR)

(21) Appl. No.: 17/280,546

(22) PCT Filed: Sep. 27, 2019
(86) PCT No.: PCT/EP2019/076142

§ 371 Date: Mar. 26, 2021

(30) Foreign Application Priority Data
Sep. 28, 2018 (FR) 1858997

(51) Int. Cl. B01L 3/00; G01N 27/49; G01N 33/18

(52) U.S. Cl. CPC B01L 3/502715; G01N 27/49; G01N 33/1813; B01L 2300/0867; B01L 2200/0684; B01L 2300/0645; B01L 2300/0819; B01L 2400/0478; B01L 2400/0666; B01L 2200/16

(57) ABSTRACT

The present invention relates to a microfluidic method for analyzing a fluid containing a metal trace element, in particular arsenic, comprising the following steps of introducing a fluid sample into at least one micro-channel of a microfluidic circuit; mixing, within the micro-channel of the microfluidic circuit, the introduced fluid sample with nitric acid and L-cysteine, and measuring the quantity of metal trace element present in the sample, using an electrochemical detection method.

### CROSS REFERENCE TO RELATED APPLICATIONS

The present application is a U.S. National Phase Application under 35 U.S.C. § 371 of International Patent Application No. PCT/EP2019/076142, filed Sep. 27, 2019, which claims priority of French Patent Application No. 1858997, filed Sep. 28, 2018. The entire contents of which are hereby incorporated by reference.

### FIELD OF THE INVENTION

The present invention relates to a method for analyzing a fluid containing a metallic trace element, for example arsenic, using a microfluidic method. The invention also concerns a microfluidic circuit, allowing the simple, automated handling of very small quantities of fluids, in particular making it possible to implement such a method.

### BACKGROUND

Arsenic is a natural element of the earth's crust that is widely present in the environment, whether in the air, water, or earth. It is highly toxic in inorganic form.

People are exposed to high levels of inorganic arsenic by drinking contaminated water, using it to prepare food, irrigating crops, during industrial processes, eating contaminated food or smoking tobacco. Prolonged exposure to inorganic arsenic, primarily through drinking contaminated water or eating food prepared with it or from crops irrigated with arsenic-rich water, can lead to chronic arsenic poisoning. Skin lesions and cancers are the most characteristic effects. Other adverse health effects that may be associated with prolonged ingestion of inorganic arsenic include developmental effects, neurotoxicity, diabetes, pulmonary and cardiovascular disease. In particular, arsenic-induced myocardial infarction may be a major cause of excess mortality (see WHO website).

The greatest threat of arsenic to public health comes from contaminated groundwater. Inorganic arsenic is naturally present at high levels in the groundwater of a number of countries, including Argentina, Bangladesh, Chile, China, India, Mexico, and the United States of America (see WHO website).

In its Guidelines for Drinking Water Quality, the WHO estimates that the current recommended limit for arsenic in drinking water is 10 μg/liter, although this value is provisional due to the difficulties of dosing and practical complications in removing it from drinking water.

Classically, the determination of arsenic in drinking water is carried out by taking samples, which are transmitted and analyzed in the laboratory. Consequently, this type of test requires multiple manipulations of the sample (especially during transport, packaging, etc.), at the risk of modifying its composition. Moreover, such tests take a long time to implement (at least a few days) before obtaining the results.

### SUMMARY

The present invention has as object the mitigation of these drawbacks of the prior art. In particular, the present invention has as object to propose a method for the analysis of a fluid containing a metal trace element (MTE), in particular arsenic, using a microfluidic method that is simple and quick to implement, but also reliable, with very good sensitivity and reproducible measurements. In particular, the method according to the invention does not denature the sample tested, and/or avoids the hydrolysis of the MTE to be analyzed. The detection limit of the species to be analyzed is compatible with the regulatory thresholds (μg/L or less). Finally, it requires a very small sample volume.

Another object of the present invention is to provide a microfluidic circuit allowing the implementation of such a method.

The invention thus relates to a microfluidic method for analyzing a fluid containing at least one metal trace element comprising the following steps:
a) introduction of a fluid sample into at least one microchannel of a microfluidic circuit;
b) mixing, within the microchannel of the microfluidic circuit, the fluid sample introduced in the step a) with reagents, and
c) measuring the quantity of metal trace element present in the sample obtained in b), using an electrochemical detection method.

Preferably, the step c) is carried out using at least 2 electrodes, preferably at least 3 electrodes, preferably at least 3 electrodes, one of which is gold. Preferably, the step c) is carried out using at least one platinum electrode as a reference electrode. Preferably, the measurement of the step c) is carried out using the following three electrodes:
one gold electrode as a working electrode,
one platinum electrode, as a reference electrode, and
one platinum counter-electrode.

Preferably, the step c) comprises mixing the sample obtained in b) with at least one solution comprising a known concentration of metal trace element (“standard solution”), and then determining the metal trace element by electrochemical detection method.

Preferably, the invention relates to a microfluidic method for the analysis of a fluid containing arsenic, comprising the following steps:
a) introducing a fluid sample into at least one microchannel of a microfluidic circuit;
b) mixing, within the microchannel of the microfluidic circuit, the fluid sample introduced in the step a), with nitric acid and L-cysteine, and
(c) measuring the quantity of arsenic present in the sample obtained in (b), using an electrochemical detection method.

Preferably, the said step c) comprises mixing the sample obtained in b) with at least one solution comprising a known concentration of metal trace element and then measuring the metal trace element by an electrochemical detection method.

Preferably the step c) is carried out using at least 2 electrodes, preferably at least 3 electrodes, preferably at least 3 electrodes, one of which is gold.

The electrode can be any electrode usable in electrochemistry, such as an electrode made of gold, possibly coated with gold nanoparticles; a thin film electrode; or an electrode made of carbon nanotubes.

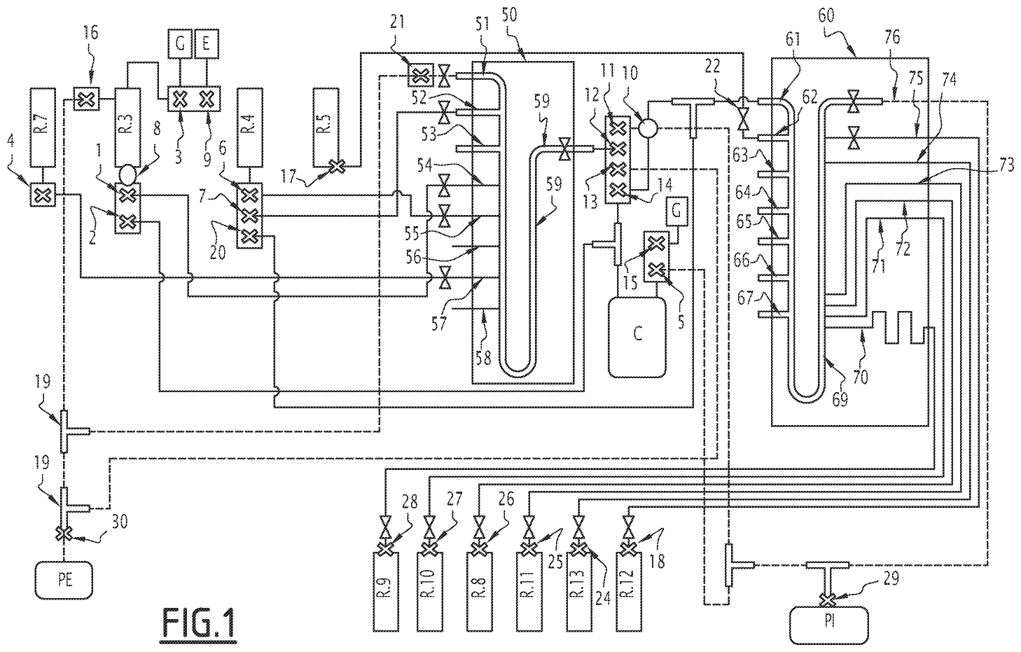

This method can be implemented particularly easily, in a single microfluidic circuit in which the different steps are carried out. This circuit is illustrated in FIG. 1.

The method according to the invention is preferably fully automated and allows the user to dispense with the various sample pre-treatment steps and analysis steps which are sometimes complex and require the handling of chemical products such as concentrated acids or standard solutions of the MTE to be analyzed.

“Fluid” means any liquid body capable of assuming the shape of the container in which it is contained. Preferably, the fluid according to the invention is a solution. Preferably, the fluid according to the invention is water. The water tested according to the method according to the invention may be any type of water.

The term “metal trace element”, also called “MTE”, means a metal that is toxic or toxic above a certain threshold. Preferably, the MTE is selected from lead, mercury, arsenic, copper, zinc, and cadmium. Preferably, the MTE according to the invention is arsenic.

Preferably, the method according to the invention is a method for analyzing water containing an MTE, in particular arsenic.

Preferably, it is implemented using a portable device including the microfluidic circuit.

The method according to the invention can also be used for the analysis of any MTE present in a solution or a trace fluid.

The use of nitric acid as a reagent in the method according to the invention, in particular for the detection of arsenic, is particularly relevant: in fact, the analysis of trace MTEs such as arsenic is most often carried out in an acid medium to minimize interference, to improve sensitivity and to avoid hydrolysis of the MTE to be analyzed or of the species formed after electrochemical transformation (D. Jagner, M. Josefson, S. Westerlung, Anal. Chem. 53 (1981) 2144; J. Lexa, K. Stulik, Talanta 30 (1983) 845; and E. Munoz, S. Palmero, Talanta 65 (2005) 613-620). Hydrochloric acid (HCl) is the most widely used because it leads to the best level of detection (LOD) (E. Munoz et al., cited above). However, this acid is known to attack the gold electrodes used to measure the quantity of MTE, thus reducing the life span of the analysis system (E. Munoz et al., cited above).

Another acid could also be used, such as hydrochloric acid, sulfuric acid or acetic acid.

However, advantageously, the use of nitric acid, in combination with L-cysteine and the confined nature of the reagents in the microfluidic channels (rapid reaction and diffusion of the species), made it possible to obtain LODs comparable to those obtained with hydrochloric acid. Moreover, unlike hydrochloric acid, the use of nitric acid allows a very large number of analyses, i.e., at least 400, preferably at least 450, preferably at least 500 analyses, to be performed without destruction of the gold electrode.

Indeed, the interest of the microfluidic circuit according to the present invention lies in the fact that it is reusable, and not disposable; it typically makes it possible to carry out at least 400 analyses, preferably at least 450, preferably at least 500 analyses, without destroying the gold electrode.

L-cysteine is a reagent that converts arsenic (V) (AsV) to arsenic (III) (AsIII) when the solution is heated. It therefore allows the speciation of arsenic, i.e., to differentiate AsIII and AsV. The transformation reaction is given below (Talanta 58 (2002) 45-55):

$${H}_{2} {AsO}_{4}^{-} + 2 {SH —CH}_{2} — {CH ( {—NH}_{2} )}_{( ctsteine )} —COOH \to {H}_{2} {AsO}_{4}^{-} + HOOC —CH ( {—NH}_{2} ) {—CH}_{2} —S—S— {CH}_{2} —CH ( {—NH}_{2} ) —COOH ( cystine ) + {H}_{2} O$$

or in a simplified way: AsV+cysteine=>AsIII+cystine.

Preferably, the method according to the invention does not use hydrazine hydrochloride (N₂H₄—HCl).

Preferably, the step c) of the method comprises mixing the sample obtained in b) with at least one solution comprising a known concentration of metal trace element (“standard solution”), and then determining the metal trace element by electrochemical detection method.

Preferably at least one standard solution is used, preferably at least two standard solutions. “Standard solution” means an MTE solution of known concentration. This concentration is determined beforehand according to the concentration of the MTE to be analyzed in the sample.

Typically, when the MTE to be measured is at a low concentration, such as strictly below 10 ppb (“low range”), two standard solutions are used, for example 2 and 4 ppb MTE.

Typically, when the MTE to be measured is at a higher concentration, such as greater than or equal to 10 ppb (“high range”), for example, between 10 and 20 ppb, two standard solutions, for example, 10 and 20 ppb MTE, are used.

The use of at least one standard solution according to the invention makes it possible to avoid matrix effects. The microfluidic method according to the invention is thus reliable and reproducible. Moreover, it can be applied to any type of fluid and does not require prior calibration. Such a method is based on dosed additions of MTE in the sample.

According to another embodiment, it is possible to set the discriminating value between the low and high range in relation to the regulatory value of the MTE to be measured. When the concentration of MTE in the sample can be measured in the low range, it is not necessary to obtain the specific concentration of the MTE: the simple fact that the concentration of MTE can be measured in the low range means that the concentration of MTE in the sample is lower than the discriminating value, and therefore lower than the regulatory value.

According to a first embodiment, the method according to the invention may include, in the step c), the detection of the MTE in the high range or in the low range. According to this first mode, typically, the concentration of the MTE is not precisely measured. The assay then includes only the detection of the MTE in the high range (concentration range greater than or equal to 10 ppb) or in the low range (concentration range less than 10 ppb).

According to a second embodiment, the method according to the invention may include, in the step c), the detection of the MTE in the high range or in the low range, and then the measurement of the concentration of the MTE within this range. The assay then includes the detection of the MTE in the high range or in the low range, and then the measurement of the concentration of the MTE within the range.

The invention also relates to a microfluidic circuit for the analysis of a fluid, in particular suitable for the implementation of the method according to the invention, comprising:
at least one storage reservoir for reagent(s), preferably nitric acid and L-cysteine, and optionally at least a second storage reservoir comprising at least one standard solution;
at least one first microfluidic chip, known as a premixing chip, comprising at least one first fluidly connected microchannel:
   at a first end, to both reservoirs and to an inlet, and
   at the second end, to a reservoir,

the said inlet suitable for injection of a sample of fluid to be analyzed; and
at least a second microfluidic chip, called an analysis chip, comprising at least a second microchannel connected to the reservoir and comprising at least two electrodes, preferably at least three electrodes, one of which is gold.

The first chip and the second chip according to the invention can also, according to an embodiment, be prepared on a single substrate. In such a case, a single chip is thus obtained, comprising a first compartment (corresponding to the premixing chip) and a second compartment (corresponding to the analysis chip).

Preferably, the microfluidic circuit comprises, in the first chip, a first microchannel array in which the fluid sample to be analyzed and the reagents (such as nitric acid and L-cysteine) circulate; and in the second chip, a second microchannel array comprising at least 2 electrodes, preferably at least 3 electrodes.

Preferably, the first microchannel array is present in a first microfluidic chip, usually called a “pre-treatment chip” or “mixing chip”, which is used to mix the different reagents (such as nitric acid and L-cysteine) with the sample, especially in specific proportions.

Preferably, the second microchannel array is present in a second microfluidic chip, generally referred to as the “analysis chip”; this is the chip on which the detection and quantification of target pollutants is carried out, thanks to the presence of the two or three electrodes, and in particular also thanks to the presence of calibration solutions (or standard solutions).

The circuit according to the invention, integrated in particular in chips as described above, allows the implementation of the method described above, in a particularly easy way.

Microfluidic chips, also called “lab-on-a-chip”, according to the Anglo-Saxon terminology sometimes used by a person skilled in the art, are miniaturized devices for biological or chemical analysis, consisting of at least one thin plate (of the order of a few tens to a few hundred micrometers), preferably consisting of glass (i.e. a hard, brittle and transparent substance with a glassy structure, essentially formed of alkali silicates, and having a high chemical resistance), and a cover comprising at least one microfluidic channel (or microchannel). Each chip is preferably as described in EP2576056.

Preferably, the chips constituting the microfluidic circuit according to the invention (i.e., premixing chip and analysis chip) each comprising:
a plate,
a cover comprising at least one microchannel, and
a single layer, intermediate between the plate and the cover, formed of an inorganic matrix of SiO2.

Preferably, the single layer has a thickness between 100 nm and 10 nm, and preferably between 300 nm and 400 nm. Preferably, each chip has at least one circuit in the cover and/or at least one circuit on the cover, associated with the cover by an inorganic matrix of SiO2.

Preferably, the microfluidic chips according to the invention are made of glass. Indeed, when the plate and the cover of the said chip are made of glass, the chips fully benefit from the exceptional properties of glass, namely:
a high optical transparency allowing good observation;
a high mechanical strength, with a high Young's modulus and a high breaking stress (depending on the type of glass used);
low porosity, which makes the chip perfectly suited to chemical analysis applications in very low concentration conditions (analysis of low-concentration toxins, for example), avoiding any pollution from outside the chip and any leakage of hazardous products to the outside;
chemical inertness to most chemical compounds (with the exception of hydrofluoric acid derivatives), for example, such as concentrated acids and non-aqueous solvents. Therefore, various chemical solutions can be circulated through the microchannels. For example, the surfaces of the fluid channels are naturally hydrophilic as a result of the chemical treatments carried out for the manufacture of the chip. This specificity is important for the analysis of biological samples. However, if necessary, it is also possible to treat the fluid channels to make them hydrophobic by circulating a suitable solution. Finally, the chip can undergo chemical cleaning, and can be biopassivated on the surface by a simple circulation of an adapted liquid known to a person skilled in the art, to obtain at will a biocompatibility. The advantage of the chemical inertness of the chip, can be seen here;
a high electrical insulation which allows in particular a correct functioning of the circuits and the application of strong external electric fields (as in the case of capillary electrophoresis on the chip for example).

The present invention will be better understood by reading the following description of preferred embodiments, given for illustrative and non-limitative purposes, and accompanied by FIG. 1, which is a plan, in top view, of a circuit comprising the microfluidic chips allowing the implementation of a method according to the invention.

### BRIEF DESCRIPTION OF THE DRAWING

### Microfluidic Circuit

FIG. 1 is a plan, in top view, of a circuit comprising the microfluidic chips allowing the implementation of a method according to a preferential embodiment of the invention. Preferably, this circuit comprising the microfluidic chips is suitable for the detection of arsenic in a fluid sample such as water. This plan shows the different microchannels that are provided within this circuit.

### DETAILED DESCRIPTION

This circuit generally consists of the following elements:
the reservoirs (R.3, R.4, R.5, R.7 and R.8 to R.13): their role is to store the different reagent solutions and the sample to be analyzed.

In particular, the reservoir R.3 contains the sample to be analyzed.

Typically, the reservoir R.4 is used to store nitric acid (HNO₃), preferably of 2.2 M concentration. The nitric acid has a double role: as mentioned above, it cleans the microfluidic circuit, but also acidifies the sample to be analyzed.

Preferably, the circuit includes a reservoir R.5, which contains a mixture of the nitric acid (especially at 100 mM) and L-cysteine (especially at 15 mM): it serves as a measurement blank, or control solution. In other words, the reservoir R.5 contains a sample devoid of MTE, which serves as a control. It is used to check that the circuit has not been contaminated by the sample to be analyzed.

The reservoir R.7 contains L-cysteine, preferably at 50 mM.

The reservoirs R.8 to R.11 preferably contain calibration solutions (or standard solutions). In particular, they contain As(III) solutions of respective concentrations equal to 14.48, 28.96, 72.40 and 144.80 ppb, acidified with 10 mM nitric acid. These solutions are used to make additions of 2 and 4 ppb As(III), or 10 and 20 ppb As(III), to the sample to be analyzed (by definition of unknown concentration), which depends on the concentration to be analyzed. For a range of concentrations to be analyzed between 0 and 10 ppb, the addition of 2 and 4 ppb solutions are used, while additions of 10 and 20 ppb solutions are used for concentrations greater than 10 ppb.

Preferably, the reservoir R.12 contains an aqueous solution of sulfuric acid (H₂SO₄), preferably at 100 mM. It allows in particular the cleaning of the working electrode of the analysis chip, by an electrochemical way between different measurements.

Finally, the reservoir R.13 preferably contains a mixture of tetrachloroauric acid (HAuCl₄), preferably at 2 mM, and sulfuric acid (H₂SO₄), preferably at 100 mM. This solution is used for automatic electrochemical regeneration of the working electrode (especially gold) of the analysis chip in case its surface deteriorates.
the internal (IWB) and external (EWB) waste bins:

The external waste bin contains the excess sample to be analyzed that has been injected into the system, or that has been used to rinse the reservoir R.3.

The internal waste bin (waste bin with gas-permeable cover), which is inaccessible to the user, contains all the solutions containing chemicals, such as acid solutions, L-cysteine solution or the mixture of tetrachloroauric acid and sulfuric acid.
the reservoir (C):

This is a recirculating reservoir with one inlet and one outlet. It is used to heat the sample to be analyzed, in the presence of L-cysteine, and thus carry out the conversion from As(V) to As(III). This heating is typically performed by a heating resistor mounted on the reservoir.
the debubblers (references 8 and 10 on FIG. 1):

The debubblers are indicated in a circle on FIG. 1. There is one between the reservoir R.3 and the solenoid valve 1 (debubbler 8); and another at the outlet of the solenoid valve 14 (debubbler 10). They are used to remove air or gas bubbles trapped in the liquid circulating in the circuit.
the solenoid valves (SVs), shown by crosses in FIG. 1:

These are electrically controlled systems, which allow the passage or not of the liquid in the circuit. Thus, when a solenoid valve is open, it will allow the liquid to pass through, while when it is closed the liquid is blocked and cannot pass through.
the connection elements (tubes, screws, unions, ferrules), shown by an hourglass as the reference 22 in FIG. 1:

These are the different elements that allow to connect the different parts of the circuit. The liquid can thus circulate in all the circuit.
a gas bottle (G):

A one liter bottle at 12 bars of nitrogen is used to generate a pressure to move the different solutions in the system. Preferably, a positive pressure of 500 mbar is used throughout the analysis. This bottle is connected to the circuit via the solenoid valve 3, which allows the gas to be injected into the circuit.
the microfluidic chips (50 and 60):
   pre-treatment chip or mixing chip (reference 50 in FIG. 1):

It allows a pre-treatment of the sample, making the different mixtures in the desired proportions. The user is thus freed from these steps, which are often long and require the handling of dangerous reagents, such as concentrated acids.

It is delimited, in FIG. 1, by the inlets 51 to 58, the inlet 51 being connected to the solenoid valve 21; and by the outlet of the main microchannel 59 (also called “first microchannel”) on the solenoid valve 12. The main microchannel 59, also called “first microchannel”, is the microchannel starting at the inlet 51 and ending at the solenoid valve 12.

Specifically, the inlet 51 is connected to the external WB via the solenoid valve 21.

The inlet 52 is connected to the reservoir R.4 (containing the nitric acid) via the solenoid valve 7.

The inlets 53, 56 and 58 are not connected. They can be used in the following cases in particular: one inlet is provided for the addition of a complexing agent, such as EDTA, to complex the metal interference potentials; another inlet is provided for diluting the sample with water, especially ultrapure water, if the sample is highly concentrated and outside the linearity range of the sensor; finally, the last inlet is suitable for a more concentrated nitric acid solution, which would be used in case of dilution, in order to bring the final pH to an acceptable value, e.g. 1.

The inlet 54 is connected to the reservoir R.3 (containing the sample to be analyzed) via the solenoid valve 1.

The input 55 is connected to the reservoir R.4 (containing nitric acid) via the solenoid valve 6.

Finally, the inlet 57 is connected to the reservoir R.7 (containing L-cysteine) via the solenoid valve 4.

This chip 50 has a channel depth of 50 μm. Typically, the length between the inlet 51 and the outlet 59 is 142 mm.

The main microchannel 59 has a width typically between 0.7 and 2 mm, preferably between 0.7 and 1.5 mm, preferably 1 mm.

The flow rate of the main microchannel is preferably 11 ml/h at a pressure of 500 mbar.

Preferably, the dimensions of the different channels of the pre-treatment chip are as follows:

| | Channel length | Channel width |
|---|---|---|
| Inlet | (in mm) | (in mm) |
| 51 | 15 | 1 |
| 52 | 15 | 1 |
| 53 | 15 | 0.404 |
| 54 | 15 | 0.400 |
| 55 | 144 | 0.285 |
| 56 | 15 | 0.380 |
| 57 | 36 | 0.276 |
| 58 | 36 | 0.391 |
| Outlet 59 | 15 | 1 |

Preferably, the dimensions of the different channels are chosen so that at the outlet of the main microchannel of the chip, the mixtures are homogeneous and in the proportions below:

| | Normalized V |
|---|---|
| V (sample) | 0.63 |
| V (HNO3 2.2M) | 0.04 |
| V (L-Cysteine 50 mM) | 0.32 |
| V total | 1.00 |
| Sample dilution | 1.58 |

Preferably, the dimensions of the mixing chip 50 are such that for a total normalized volume leaving the chip (equal to 1), the volume of the sample to be analyzed is 0.63, the volume of nitric acid is 0.04 and the volume of L-cysteine is 0.32. In this case, we have a sample dilution of 1.58 (=1/0.63).
analysis chip 60:

This chip 60 comprises a three-electrode system, on which the mixtures leaving the pre-treatment chip 50 are analyzed.

The analysis chip 60 is delimited, in FIG. 1, by the inlets 61 to 67, with the inlet 61 being connected to a debubbler 10 and the solenoid valve 14; by the inlets 70 to 75; and by the outlet of the main microchannel 69 (also called the “second microchannel”) in the internal WB.

The main microchannel 69 has a width typically between 0.7 and 2 mm, preferably between 0.7 and 1.5 mm, preferably equal to 1 mm.

The analysis chip 60 typically has 13 inlets (references 61 to 67 and 70 to 75) and one outlet 76. These inlets include inlets 70 to 75 connected to the reservoirs R.8 to R.13, and the inlets 61 to 67.

The inlet 61 is connected to a debubbler 10, the solenoid valve 14 and the reservoir R.4 (containing nitric acid) via the solenoid valve 20.

The inlet 62 is connected to the reservoir R.5 (containing the measurement blank) via the solenoid valve 17.

The inlets 63 to 67 can be connected to ultrapure water reservoirs and/or for the addition of buffer solutions, such as acetates or phosphates. They allow measurements to be carried out on highly concentrated samples, or at pH values close to the pH value of drinking water, i.e., without acidification.

In addition, the reservoirs R.8 to R.11 (containing the calibration solutions) are connected to the main microchannel 69 via the solenoid valves 26, 28, 27 and 25 respectively.

The reservoir R.12 (containing the aqueous sulfuric acid solution) is connected to the main microchannel 69 via the solenoid valve 18.

Finally, the reservoir R.13 (containing the mixture of tetrachloroauric acid and sulfuric acid) is connected to the main microchannel 69 via the solenoid valve 24.

In detail, preferably, the three-electrode system includes:
a gold working electrode with a size of 1.06 mm×1 mm,
a platinum reference electrode with a size of 2.96 mm×1 mm, and
a platinum counter-electrode with a size of 6.74 mm×1 mm.

all electrodes being located in the main microchannel 69 (not shown in FIG. 1).

The depth (or width) of the analysis chip 60 is 20 μm. The length between the inlet 61 and the outlet 76 is typically 178 mm, and the width of the main microchannel 69 is 1 mm.

Preferably, the average outlet flow rate is about 400 μl/h at a pressure of 500 mbar.

Typically, the sample from the pre-treatment chip enters through one of the channels of the pre-treatment chip, for example, the inlet 61, as does the nitric acid from the reservoir R.4.

Another channel (i.e., the inlet 62 via the solenoid valve 17) is dedicated to measuring the blank (reservoir R.5) (control solution), which serves as a control to ensure that there is no contamination from the system.

The dimensions of the different channels of the analysis chip necessary to obtain the ratios allowing a satisfactory LOD are as follows:

| | Channel length | Channel width |
|---|---|---|
| Inlet | (in mm) | (in mm) |
| 61 | 10 | 1 |
| 62 | 10 | 0.862 |
| 63 | 10 | 0.748 |
| 64 | 10 | 0.190 |
| 65 | 10 | 0.622 |
| 66 | 10 | 0.446 |
| 67 | 10 | 0.344 |
| 70 | 50 | 0.152 |
| 71 | 50 | 0.150 |
| 72 | 52 | 0.150 |
| 73 | 54 | 0.150 |
| 74 | 10 | 1 |
| 75 | 10 | 1 |
| 76 | 10 | 1 |

Typically, each chip (pre-mixing chip and/or analysis chip) can be composed of two superimposed plates, glued together. Thus, each chip can be composed of a first plate, which can for example be a transparent microscope slide, and a second plate whose face in contact with the first plate is engraved so as to define microchannels between the two plates which are superimposed and glued to each other. The first plate can be made of a polymer material. The material constituting at least one of the two plates may be transparent. The dimensions of the microchannels are determined by adapting the width and depth of the engravings in the engraved plate. It should be noted that microfluidic chips manufactured according to other methods known to the man skilled in the art can obviously be used to implement the invention.

As a complement to the elements mentioned above, the microfluidic circuit according to the invention can be connected especially to at least one element chosen among an electronic device necessary for the operation of the system, a battery, a potentiostat for piloting the electrochemical measurements, a cooling system placed on the chips to cool the solutions coming from the reservoir and a screen, in particular a touch screen, which makes it possible to launch the desired measurement, to know the state of progress of the measurement and to visualize the result obtained.

### Microfluidic Method

As previously indicated, the invention relates to a microfluidic method for analyzing a fluid containing at least one MTE comprising the following steps:
a) introducing a fluid sample into at least one microchannel of a microfluidic circuit;
b) mixing, within the microchannel of the microfluidic circuit, the fluid sample introduced in step a) with reagents, and
c) measuring the quantity of MTE present in the sample obtained in b), using an electrochemical detection method.

Preferably the step c) is carried out using at least 2 electrodes, preferably at least 3 electrodes, preferably at least 3 electrodes, one of which is gold.

Preferably, the invention relates to a microfluidic method for the analysis of a fluid containing arsenic, comprising the following steps:
a) introduction of a fluid sample into at least one microchannel of a microfluidic circuit;
b) mixing, within the microchannel of the microfluidic circuit, the fluid sample introduced in the step a), with nitric acid and L-cysteine, and
(c) measuring the quantity of arsenic present in the sample obtained in (b), using an electrochemical detection method.

Preferably the step c) is carried out using at least 2 electrodes, preferably at least 3 electrodes, preferably at least 3 electrodes, one of which is gold.

The step a) of introducing a fluid sample into at least one microchannel of a microfluidic circuit is preferably performed according to the following sub-steps:
a1) injection of the sample into the microfluidic circuit; then
a2) pressurizing the sample in the circuit.

The injection of the sample into the microfluidic circuit (sub-step a1) is carried out in particular by injecting the said sample into the inlet of the first microchannel of the first chip of the circuit according to the invention. In particular, this step is carried out using a syringe equipped with a 0.45 μm filter. The filter removes all suspended matter with a diameter greater than 0.45 μm.

More precisely, referring to FIG. 1, initially the solenoid valves 9, 16 and 30 are opened. The solenoid valve 9 allows the sample (S) to pass through to the reservoir R.3. Part of this sample is used to rinse the reservoir and goes to the external WB through the solenoid valves 16 and 30, whereas the rest of the sample remains in the reservoir R.3 and is used for analysis.

Typically, this operation may take a few minutes or seconds and then the solenoid valves 9, 16 and 30 are closed.

The said sample is then pressurized (sub-step a2). Pressurization of the sample can be carried out by any means, for example by injecting a gas, especially an inert gas, or by suction. For example, the pressurization can be carried out by a pump or a syringe. By pressurizing the sample, the sample is set in motion.

For example, the sample is stored in a reservoir (R.3) connected to a microchannel of the microfluidic circuit. Preferably, it is stored in a reservoir (R.3) connected to the first microchannel of the first chip and pressurization of the reservoir R.3 is carried out in particular by opening a solenoid valve (the solenoid valve 3), which is connected to the nitrogen gas bottle (G).

Preferably, the other reservoirs, except the reservoir R.3, are always under pressure during the whole method (i.e., before and after the measurement). The solenoid valve 3 remains open for the duration of the analysis; it is closed at the end of the measurement.

Between the steps a) and b) of the method according to the invention, a step N) can be carried out: this is the cleaning step.

This step N preferably comprises at least one, preferably at least two, preferably at least three, preferably the following four sub-steps:
N1: a sub-step for cleaning the microfluidic circuit; and/or
N2: a sub-step for cleaning the measuring electrodes, especially the gold electrode; and/or
N3: a sub-step of electrochemical gold deposition; and/or
N4: a control sub-step, especially by measuring a control solution.

Preferably, the sub-step N1 is performed as follows:

The solenoid valves 1, 21 and 30 are open. The sample inlet channel in the pre-treatment chip 50 (inlet 54 of the pre-treatment chip) is cleaned with the sample, then the sample fraction used for cleaning is returned to the external waste bin, in particular through the solenoid valves 21 and 30. This operation typically lasts about 30 seconds, after which the solenoid valves are closed.

Then, the solenoid valves 2, 5 and 29 are opened. The sample is pushed towards the reservoir especially through the solenoid valve 2, and then into the internal waste bin, especially through the solenoid valves 5 and 29. This operation typically lasts 30 seconds and then the solenoid valves are closed.

Then the reservoir is emptied, in particular through solenoid valves 15, 13 and 30. The solenoid valve 15 sends the gas G (as with solenoid valve 3) into the reservoir; the gas then exerts pressure on the liquid contained in the reservoir and pushes it towards the external waste bin, in particular by means of the solenoid valves 13 and 30. This operation typically lasts 15 seconds, then the solenoid valves are closed.

Preferably, sub-step N2 is performed as follows:

Preferably, at least one electrode used in the step c) of the method according to the invention, called the working electrode, is made of gold. In this case, it is preferable to clean it before any measurement, in order to eliminate oxides potentially formed on its surface over time, or to eliminate traces of MTE (arsenic in particular) remaining on the electrode from the previous measurement.

For this purpose, sulfuric acid (contained in the reservoir R.12) is used to clean the gold electrode present in the main microchannel 69 of the analysis chip 60, in particular by cyclic voltammetry (voltammetry). The solenoid valves 18 and 29 are open to let this acid through. Cycling is typically performed between −0.4 and 1.5 V at 200 mV/s. This operation usually lasts 3 minutes, then the solenoid valves are closed.

Preferably, sub-step N3 is performed as follows:

The object of this sub-step N3 is to increase the electrochemically active surface of the gold, obtained by electrochemical deposition under vacuum.

The measuring surface will then no longer be flat, but in relief (in 3D), because the electrochemical deposition leads to a non-planar surface. To do this, generally the solenoid valves 24 and 29 are opened for about 3 minutes, the mixture of tetrachloroauric acid and sulfuric acid from the reservoir R.13 is then released in the inlet 74 and then in the main microchannel 69, and the gold deposit is made by chronoamperometry for about 300 seconds at the peak potential of Au(III) deposit on the working electrode. This potential is determined by cyclic voltammetry. Gold deposition can also be achieved when after a certain number of measurements, the active gold surface is reduced, resulting in a reduction of the peak area of gold oxide reduction by cyclic voltammetry measurement. In this case, the system automatically initiates a gold deposit for regeneration.

Preferably, sub-step N4 is carried out as follows:

Measuring the control solution (blank) can be carried out to check the cleanliness of the previously cleaned circuit. For this purpose, solenoid valves 17 and 29 are usually open for about 5 minutes. The analysis of the blank (control solution) (contained in reservoir R.5) is typically done by SWV (Square Wave Voltammetry).

The content of the reservoir R.5 is released into inlet 62 and then into the main microchannel 69, to be measured, before being disposed of in the internal waste bin.

The analysis of the blank is done with a deposition potential (Edep) of −1.1 V, for 90 seconds (Tdep), with an amplitude of 0.02 V. The signal is recorded between −0.2 and 0.7 V. If a peak appears, then the sub-steps N1 to N3 are restarted, preferably automatically, otherwise one proceeds to the step b) of the method according to the invention.

Once the sample has been introduced, and the possible cleaning step(s) carried out, the second step (step b) follows: the mixing, within the microchannel of the microfluidic circuit, of the sample with reagents, preferably nitric acid and L-cysteine.

Typically, during this step b), the two reagents, especially present in the two reservoirs fluidly connected to one end of the first chip, are released in the first microchannel of the said first chip and mix with the sample injected into the inlet.

Preferably, the mixture obtained is then conveyed into a reservoir, preferably into the reservoir connected to the second end of the first microchannel of the first chip.

Preferably, the first microchannel of the first chip is the main microchannel (microchannel 59 in FIG. 1) and has a width typically between 0.7 and 2 mm, preferably between 0.7 and 1.5 mm, preferably equal to 1 mm; and a length typically between 30 and 60 μm, preferably between 40 and 55 μm.

Preferably, the microchannel flow rate of the first chip is 11 ml/hour at a pressure of 500 mbar.

Typically, in this step b), solenoid valves 4, 1, 5, 6, 12 and 29 are opened. The sample, nitric acid and L-cysteine are then mixed in the desired proportions using the mixing chip 50 and fed into the reservoir through solenoid valve 12. This operation usually takes a few minutes, typically 2 to 5 minutes, preferably 2 to 3 minutes, more preferably 2 minutes and 15 seconds, and then the solenoid valves are closed.

Then the connecting tubes and the reservoir are rinsed with the pre-treated sample.

Then the solenoid valves 11, 15 and 29 are opened, preferably for 15 seconds. The pre-treated sample is then sent to the internal waste bin.

The reservoir is filled again by opening solenoid valves 4, 1, 5, 6, 12 and 29, usually for a few minutes, typically 2 to 5 minutes, preferably 2 to 3 minutes, more preferably 2 minutes and 15 seconds, and then the pretreated sample is sent, this time to the analysis chip 60 by opening solenoid valves 14, 15 and 29.

Preferably, the dimensions of the different channels are chosen so that at the outlet of the main microchannel 59 of the chip 50, the mixtures are homogeneous and in specific proportions.

In particular, the mixture of sample, nitric acid (at 2.2 mM) and L-cysteine (at 50 mM) is made in a volume ratio of 0.6-0.7:0.03-0.05:0.25-0.40 respectively. Preferably, this respective volume ratio is equal to 0.63:0.04:032.

Finally, once the sample has been mixed with reagents, preferably nitric acid and L-cysteine, the method comprises a step c) of measuring the quantity of MTE present in the sample obtained in b), using at least 3 electrodes, one of which is gold.

The step c) preferably comprises circulating the sample obtained in b) from the reservoir through the second microchannel of the analysis chip, comprising at least three electrodes, one of which is gold.

Preferably, the second microchannel of the second chip (analysis chip) is the main microchannel (microchannel 69 in FIG. 1), and has a width typically between 0.1 and 2 mm, preferably between 0.12 and 1.5 mm; and a length typically between 5 and 80 mm, preferably between 9 and 60 mm.

Typically, in the step c), the sample obtained in b) (also called pre-treated sample), once in the analysis chip 60, is analyzed using at least 3 electrodes, one of which is gold.

Preferably, in the step c), at least one standard solution is mixed with the sample obtained in b).

More specifically, in the case of arsenic, during the step c), the pre-treated sample is analyzed using at least 3 electrodes, one of which is gold, and according to the following sub-steps:
c1) measurement of the quantity of arsenic (III) present in the sample, called As(III), then
c2) conversion of the arsenic (V) remaining in the sample to arsenic (III), then measuring the quantity of arsenic (III) obtained, called As tot, and finally
c3) determining the amount of arsenic actually present in the sample by the formula As(V)=As tot−As(III).

First of all, the amount of As(III) is determined; this is step c1).

Preferably, this step c1) involves at least one standard solution. Preferably, the step c1) involves mixing the sample obtained in b) with at least one standard solution, preferably two standard solutions of different concentrations, and then determining the concentration of As(III) present in the sample. “Standard solution” means a solution comprising a known concentration of As(III). For example, a first standard solution with an As(III) concentration between 5 and 15 ppb and a second standard solution with an As(III) concentration between 15 and 25 ppb can be used; or a first standard solution with an As(III) concentration between 1 and 3 ppb and a second standard solution with an As(III) concentration between 3 and 5 ppb can be used.

Preferably, the quantity of As(III) is measured by circulating the sample obtained in b) by SWV, especially with the same electrochemical parameters as the control solution (blank), with the exception of Tdep (deposition time), i.e., a deposition potential (Edep) of −1.1 V, for 120 seconds (Tdep), with an amplitude of 0.02 V, and the signal is recorded between −0.2 and 0.7 V.

The average area (Amoy) of the peak measured during the desorption of arsenic on the gold electrode is compared with threshold values.

In particular:
if 3×10⁻³ μAV<Amoy<3×10⁻² μAV, then the system opens the solenoid valves 27 and 25, which provide additive concentrations (standard solutions) in the mixture of 10 and 20 ppb respectively (i.e., reservoirs R.10 and R.11). At each addition, the Amoy (addition) is measured. Thus, the values of Amoy, Amoy (addition 1) and Amoy (addition 2) are used to determine the concentration of As(III) in the sample;
if Amoy <3×10⁻³ μAV, then the measurement of Amoy is repeated using a deposition time of 360 seconds. In this case, if the resulting signal gives an Amoy <3×10⁻⁴ μAV, then the sample contains As(III) in a concentration below the limit of quantification of 0.85 ppb. Conversely, if Amoy >3×10⁻⁴ μAV, then the system opens the solenoid valves 26 and 28, which provide additive concentrations in the mixture of 2 and 4 ppb respectively (i.e., reservoirs R.8 and R.9). At each addition, the Amoy (addition) is measured. Thus, the values of Amoy, Amoy (addition 1) and Amoy (addition 2) are used to determine the concentration of As(III) in the sample;
if Amoy >3×10⁻² μAV, the sample is automatically diluted at the pre-treatment chip 50, depending on the measured Amoy. Then a Tdep of 360 seconds or 120 seconds is applied.

Then the amount of As(V) is determined; this is step c2).

The measurement of As(V) is obtained indirectly by subtracting the real As(III) content from the total arsenic content (As tot) (i.e., As(V)=As tot−As(III)). The total arsenic (As tot) is obtained by converting all As(V) to As(III).

To convert arsenic (V) to arsenic (III), preferably an incubation step of the sample obtained in b) takes place in the reservoir, preferably by heating. Typically, this incubation step is carried out for a few minutes.

For this purpose, when the mixture is in the reservoir, the solenoid valves are closed and the reservoir is heated with the heating resistor, for example, for 10 minutes. In this way, all the As(V) is transformed into As(III), which, combined with the As(III) already present in the sample, gives the total amount of arsenic. This mixture is then fed into the analysis chip 60 as previously described (i.e., by opening the solenoid valves 14, 15 and 29), to be dosed. The analysis chip 60, which has a cooling system, allows the mixture to be cooled down to around 22-23° C., and then the As(III) measurement is carried out in the same way as described above.

Finally, typically, after analysis, the solenoid valves 7, 12, 5 and 29 are opened, especially for 30 seconds, to clean the reservoir and the microchannels containing nitric acid. The nitric acid remaining in the reservoir is used to clean the analysis chip 60, typically by opening the solenoid valves 14, 15 and 29, for about 30 seconds. This chip is cleaned again and then filled with nitric acid for storage, typically for 30 minutes, by opening the solenoid valves 20 and 29.

The invention is now illustrated by the following example.

### Example 1: Implementation of the Method According to the Invention for Measuring the Level of Arsenic in a Water Sample

Analyses of arsenic-doped water samples were conducted. The choice was made between ultrapure water and two mineral waters (Volvic and Evian).

Before analysis, these waters were pre-tested by ICP-MS to ensure that they did not contain arsenic, or that the quantity of arsenic contained was below the limit of quantification of the instrument used (0.1 ppb). Subsequently, these waters to be analyzed are spiked with 10 ppb of As(III) to obtain an ultrapure water containing 10 ppb of As(III) and two samples of mineral water each containing 10 ppb. The choice of 10 ppb of As(III) is relative to the WHO standard that sets the threshold for arsenic in drinking water at 10 ppb. As(III) is chosen in this test because it is the most toxic form of arsenic.

For the analysis, 40 mL of water sample with As(III) is injected with a syringe into the microfluidic analysis system according to the invention. The method according to the invention is implemented for the analysis of these samples in turn on the glass chip. The response of the sensors is recorded in triplicate for each sample and the results below are obtained:

| | | | Ultrapure |
|---|---|---|---|
| Sample | Volvic | Evian | Water |
| As (III) | 10.00 | 10.00 | 10.00 |
| added | | | |
| (ppb) | | | |
| As (III) | 9.34 ± 0.39 | 10.67 ± 0.56 | 9.77 ± 0.44 |
| Measured | | | |
| (ppb) | | | |

The introduced arsenic is almost completely detected by the method described here.

In conclusion, the method according to the invention can be effectively used for the automatic detection of MTE in water.

1. A microfluidic method for analyzing a fluid containing at least one metal trace element comprising the following steps:
a) introduction of a fluid sample into at least one microchannel of a microfluidic circuit;
b) mixing, within the microchannel of the microfluidic circuit, the fluid sample introduced in step a) with the reagents, and
c) measuring the quantity of the metal trace element present in the sample obtained in b), using an electrochemical detection method,
the said step c) comprising mixing the sample obtained in b) with at least one solution comprising a metal trace element of known concentration, and then assaying the metal trace element by electrochemical detection method.

2. The microfluidic method according to claim 1, wherein the metal trace element is arsenic, and in that the method comprises the following steps:
a) introducing a fluid sample into at least one microchannel of a microfluidic circuit;
b) mixing, within the microchannel of the microfluidic circuit, the fluid sample introduced in the step a), with nitric acid and L-cysteine, and
c) measuring the quantity of arsenic present in the sample obtained in b), using an electrochemical detection method, preferably with, at least 2 electrodes, preferably at least 3 electrodes, one of which is gold.

3. The microfluidic method according to claim 1, wherein the measurement in the step c) is carried out using the following three electrodes:
a gold electrode as a working electrode,
a platinum electrode, as a reference electrode, and
a platinum counter-electrode.

4. The microfluidic method according to claim 1, wherein step a) is carried out according to the following sub-steps:
a1) injection of the sample into the microfluidic circuit; and
a2) pressurization of the sample in the circuit.

5. The microfluidic method according to claim 2, wherein steps a) and b) a cleaning step N) is carried out, preferably comprising at least one, preferably at least two, preferably at least three, preferably the following four sub-steps:
N1: a sub-step for cleaning the microfluidic circuit; and
N2: a sub-step for cleaning the electrodes, especially the gold electrode; and
N3: a sub-step of electrochemical gold deposition; and
N4: a control sub-step, especially by measuring a control solution.

6. The microfluidic method according to claim 1, wherein the microfluidic circuit comprises:
at least one storage reservoir for reagent(s), preferably nitric acid, and L-cysteine, and optionally at least a second storage reservoir comprising at least one standard solution;
at least one first microfluidic chip, called premixing chip, comprising at least one first microchannel fluidly connected:
at a first end, to both reservoirs and to an inlet, and
at the second end, to a reservoir,
the said inlet suitable for sample injection; and
at least a second microfluidic chip, called an analysis chip, comprising at least a second microchannel connected to the reservoir and comprising at least two electrodes, preferably at least three electrodes, one of which is gold.

7. The microfluidic method according to claim 6, wherein it comprises two reservoirs for storing two distinct reagents and in that, during the step b), the two reagents present in the two reservoirs connected to one end of the first chip are released into the first microchannel of the said first chip, and are mixed with the sample injected into the inlet, and preferably the mixture obtained is then sent into a reservoir, preferably into the reservoir connected to the second end of the first microchannel of the first chip.

8. The microfluidic method according to claim 2, wherein the mixture of the sample, nitric acid used at 2.2 mM and L-cysteine used at 50 mM from the step b) is carried out in a respective volume ratio of 0.6-0.7:0.03-0.05:0.25-0.40, preferably this respective volume ratio is equal to 0.63:0.04:0.32.

9. The microfluidic method according to claim 2, wherein step c) comprises:
c1) measuring the quantity of arsenic (III) present in the sample, called As(III),
c2) conversion of the arsenic (V) remaining in the sample to arsenic (III), then measuring the quantity of arsenic (III) obtained, called As tot, and
c3) determining the amount of arsenic actually present in the sample by the formula As(V)=As tot−As(III).

10. The microfluidic method according to claim 1, wherein step c) comprises mixing the sample obtained in b) with at least two solutions each comprising a known concentration of the metal trace element, and then determining the metal trace element assay by an electrochemical detection method.

11. The microfluidic method according to claim 10, wherein the two solutions each comprising a known concentration of metal trace element have a concentration of less than 10 ppb, for example, 2 and 4 ppb; or a concentration greater than or equal to 10 ppb, for example, 10 to 20 ppb, for example, 10 to 20 ppb.

12. The microfluidic method according to claim 11, wherein the determination of the metal trace element assay comprises only its detection in the range of concentrations less than 10 ppb or in the range of concentrations greater than or equal to 10 ppb.

13. A microfluidic circuit for analyzing a fluid, in particular suitable for implementation of the method according to claim 2, comprising:
at least two storage reservoirs for nitric acid and L-cysteine;
at least a first chip, called a premixing chip, comprising at least a first fluidly connected microchannel:
at a first end, to both reservoirs and to an inlet, and
at the second end, to a reservoir,
the said inlet suitable for injection of a sample of fluid to be analyzed; and
at least a second chip, called an analysis chip, comprising at least a second microchannel connected to the reservoir and comprising at least three electrodes, one of which is gold.
